(19) United States
(12) Patent Application Publication
(10) Pub. No.: US 2013/0116535 A1
(43) Pub. Date: May 9, 2013

(54) APPARATUS AND METHOD FOR DIAGNOSING A LESION

(71) Applicants: Jae-Cheol Lee, Seoul (KR); Yeong-Kyeong Seong, Suwon-si (KR); Kyoung-Gu Woo, Seoul (KR)

(72) Inventors: Jae-Cheol Lee, Seoul (KR); Yeong-Kyeong Seong, Suwon-si (KR); Kyoung-Gu Woo, Seoul (KR)

(73) Assignee: SAMSUNG ELECTRONICS CO., LTD., Suwon-si (KR)

(21) Appl. No.: 13/453,254

(22) Filed: Apr. 23, 2012

(30) Foreign Application Priority Data
Nov. 4, 2011 (KR) 10-2011-0114774

(51) Int. Cl. A61B 5/02; A61B 5/026; A61B 6/00

(52) U.S. Cl. 600/407

(57) ABSTRACT

An apparatus and method for diagnosing a lesion are provided. The apparatus includes an acquisition unit configured to acquire first blood-vessel information regarding a blood vessel from an image including the blood vessel, and an extraction unit configured to extract one or more tissue regions from the image based on the first blood-vessel information.

### CROSS-REFERENCE TO RELATED APPLICATION

This application claims the benefit under 35 U.S.C. §119(a) of Korean Patent Application No. 10-2011-0114774 filed on Nov. 4, 2011, in the Korean Intellectual Property Office, the entire disclosure of which is incorporated herein by reference for all purposes.

### BACKGROUND

1. Field

The following description relates to detection of a lesion from an image.

2. Description of the Related Art

With the advancement of surgery techniques, different kinds of minimum invasive surgeries have been developed. A minimum invasive surgery process represents a surgery method in which a medical operation may be performed by approaching a lesion using surgical instruments without incising skin and muscle tissues. The surgical instruments may include a syringe or a catheter. The medical operation may include a medicine injection, removal of lesions, appliance insertion etc. In order to perform the minimum invasive surgery process, doctors needs to locate the lesion. Also, in order to diagnose a disease, the doctors may need to determine the size, shape and location of the lesion.

Various medical imaging equipment has been developed that can aid in the detection of the size, shape, and location of the lesion. This medical imaging equipment includes a Computed Tomography (CT) system, a Magnetic Resonance Imaging (MRI) system, a Positron Emission Tomography (PET) system, a Single Photon Emission Computed Tomography (SPECT), etc.

However, it may be difficult to precisely extract a lesion since the images produced by the aforementioned medical imaging equipment is typically of poor quality. Accordingly, a need exists for a technology capable of precisely extracting a lesion.

### SUMMARY

In one general aspect, there is provided an apparatus for diagnosing a lesion, including an acquisition unit configured to acquire first blood-vessel information regarding a blood vessel from an image including the blood vessel, and an extraction unit configured to extract one or more tissue regions from the image based on the first blood-vessel information.

A general aspect of the apparatus may further provide that the extraction unit is further configured to compare the acquired first blood-vessel information with second blood-vessel information from storage to determine the one or more tissue regions to be extracted, the second blood-vessel information being blood-vessel information concerning a plurality of types of tissue regions.

A general aspect of the apparatus may further provide that, if the image is a breast image, if the image is a breast image, the one or more extracted tissue regions includes a subcutaneous fat tissue region, a mammary glandular tissue region, a pectoralis muscle region, or any combination thereof.

A general aspect of the apparatus may further provide a detection unit configured to detect a lesion from the one or more extracted tissue regions.

A general aspect of the apparatus may further provide that the detection unit is further configured to compare the acquired first blood-vessel information with third blood-vessel information from storage to detect the lesion, the third blood-vessel information being blood-vessel information concerning a plurality of types of lesions.

A general aspect of the apparatus may further provide a setting unit configured to set one or more of the one or more tissue regions as a lesion detection target region, and a detection unit configured to detect a lesion from the lesion detection target region.

A general aspect of the apparatus may further provide that, if the image is a breast image, the lesion detection target region includes a mammary glandular tissue region.

A general aspect of the apparatus may further provide that the acquisition unit is further configured to partition the image into a plurality of regions of a predetermined size, and acquire the first blood-vessel information according to the partitioned regions, and the extraction unit is further configured to compare the acquired first-blood vessel information with second blood-vessel information from storage to determine the one or more tissue regions to be extracted, the second blood-vessel information being blood-vessel information concerning a plurality of types of tissue regions.

A general aspect of the apparatus may further provide that the acquisition unit is further configured to partition the image into a plurality of regions of a predetermined size, and calculate a ratio of blood vessels to an area of each of the partitioned regions as a distribution of the blood vessels.

A general aspect of the apparatus may further provide that the acquisition unit is further configured to partition the image into a plurality of regions of a predetermined size, and calculate a blood flow per unit time within each of the partitioned regions as blood flow information.

A general aspect of the apparatus may further provide that the first blood-vessel information includes blood-vessel distribution information, blood-vessel location information, blood flow information, or any combination thereof.

In another general aspect, there is provided an apparatus for diagnosing a lesion, including an acquisition unit configured to acquire first blood-vessel information regarding a blood vessel from an image including the blood vessel, and a detection unit configured to detect a lesion from the image based on the first blood-vessel information.

Another general aspect of the apparatus may further provide that the detection unit is further configured to compare the acquired first blood-vessel information with third blood-vessel information from storage to detect the lesion, the third blood-vessel information being blood-vessel information concerning a plurality of types of lesions.

Another general aspect of the apparatus may further provide that the first blood-vessel information includes blood-vessel distribution information, blood-vessel location information, blood flow information, or any combination thereof.

In yet another general aspect, there is provided a method for diagnosing a lesion, including acquiring first blood-vessel information regarding a blood vessel from an image including the blood vessel, and extracting one or more tissue regions from the image based on the first blood-vessel information.

A general aspect of the method may further provide that the extracting of the one or more tissue regions includes comparing the acquired first blood-vessel information with second blood-vessel information from storage, the second blood-vessel information being blood-vessel information concerning a plurality of types of tissue regions, and determining, from the comparing, the one or more tissue regions to be extracted.

A general aspect of the method may further provide that the extracting of the one or more tissue regions includes, if the image is a breast image, extracting a mammary glandular tissue region.

A general aspect of the method may further provide detecting a lesion from the one or more tissue regions.

A general aspect of the method may further provide that the detecting of the lesion includes comparing the acquired first blood-vessel information with third blood-vessel information from storage, the third blood-vessel information being blood-vessel information concerning a plurality of types of regions, and detecting, from the comparing, the lesion.

A general aspect of the method may further provide setting one or more of the one or more tissue regions as a lesion detection target region, and detecting a lesion from the lesion detection target region.

Other features and aspects may be apparent from the following detailed description, the drawings, and the claims.

### BRIEF DESCRIPTION OF THE DRAWINGS

FIG. 1 is a diagram illustrating an example of an apparatus for diagnosing a lesion.

FIGS. 2A to 2F are diagrams illustrating an example explaining how a lesion diagnosis apparatus detects a tissue region and a lesion.

FIG. 3 is a diagram illustrating another example of an apparatus for diagnosing a lesion.

FIGS. 4A, 4B, and 4C are diagrams illustrating another example explaining how a lesion diagnosis apparatus detects a tissue region and a lesion.

FIG. 5 is a flowchart illustrating an example of a method for diagnosing a lesion.

Throughout the drawings and the detailed description, unless otherwise described, the same drawing reference numerals will be understood to refer to the same elements, features, and structures. The relative size and depiction of these elements may be exaggerated for clarity, illustration, and convenience.

### DETAILED DESCRIPTION

The following description is provided to assist the reader in gaining a comprehensive understanding of the methods, apparatuses, and/or systems described herein. Accordingly, various changes, modifications, and equivalents of the methods, apparatuses, and/or systems described herein will be suggested to those of ordinary skill in the art. Also, descriptions of well-known functions and constructions may be omitted for increased clarity and conciseness.

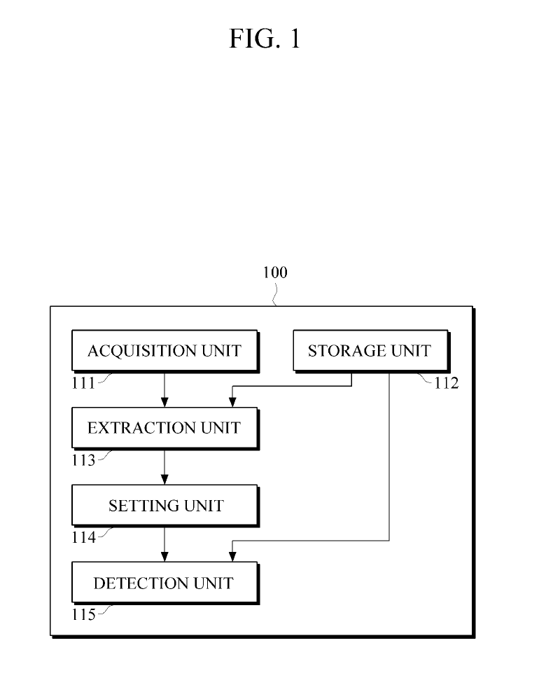

FIG. 1 is a diagram illustrating an example of an apparatus 100 for diagnosing a lesion.

Referring to FIG. 1, apparatus 100 includes an acquisition unit 111, a storage unit 112, an extraction unit 113, a setting unit 114, and a detection unit 115.

The acquisition unit 111 may acquire first blood-vessel information about a blood vessel from a medical image that contains blood vessels.

The acquisition unit 111 may be an apparatus capable of acquiring an image containing a blood vessel using angiography, Doppler sonography, computed tomography (CT), magnetic resonance imaging (MRI), etc.

Blood-vessel information may be a variety of information that relates to blood vessels, such as distribution of blood vessels, locations of each blood vessel, blood flow, and the like.

The acquisition unit 111 may partition the medical image into a plurality of regions of a predetermined size, and acquire the first blood-vessel information from each region. For example, if the medical image is partitioned into 10 regions, there may be present 10 pieces of first blood-vessel information.

In addition, the acquisition unit 111 may acquire information on the distribution of blood vessels by calculating a ratio of blood vessels to the area of each partitioned region. For example, in a two-dimensional image, the partitioned region of a predetermined size may be represented by pixels, which may be, for example, 3*3 pixels. As another example, in a three-dimensional image, the partitioned region of a predetermined size may be represented by voxels. As such, the size of the region may be varied.

The acquisition unit 111 may acquire blood flow information by calculating a blood flow per hour within each partitioned region. For example, under the assumption that each of the partitioned regions is represented by voxels and a unit time is one second, the acquisition unit 111 may acquire blood flow information by calculating a blood flow per one second within one voxel.

The acquisition unit 111 may acquire blood vessel location information based on location information of the partitioned region (for example, pixels or voxels). For example, if a blood vessel is included in a partitioned region located at the third row and the fourth column of the entire image, the blood vessel location information may correspond to the location information of the partitioned region at the third row and the fourth column.

The storage unit 112 may store a number of pieces of information about blood vessels. For example, the storage unit 112 may store second blood-vessel information directed to types of tissue regions. In other words, the second blood-vessel information may include information about blood vessels that are present in each of the types of tissue regions. For example, the storage unit 112 may store blood-vessel information about the blood vessels that are included in a first type of tissue region and blood-vessel information about the blood vessels that are included in a second type of tissue region.

The storage unit 112 may store third blood-vessel information of each type of lesion. The third blood-vessel information may include information about a blood vessel that is included in each type of lesion. For example, the storage unit 112 may include blood-vessel information about a blood vessel that is included in a first type of lesion and blood-vessel information about a blood vessel that is included in a second type of lesion.

The storage unit 112 may be at least one of a variety of storage media including flash memory type, hard disk type, multimedia card micro type and card-type memories (for example, SD or XD memory), and RAM, ROM, and web storage.

The extraction unit 113 may extract at least one tissue region from the image based on the first blood-vessel information acquired by the acquisition unit 111. For example, the extraction unit 113 may compare the first blood-vessel information, which is acquired by the acquisition unit 111, with the second blood-vessel information present in the storage unit 112 regarding each type of tissue region, and extract at least one tissue region based on the comparison result.

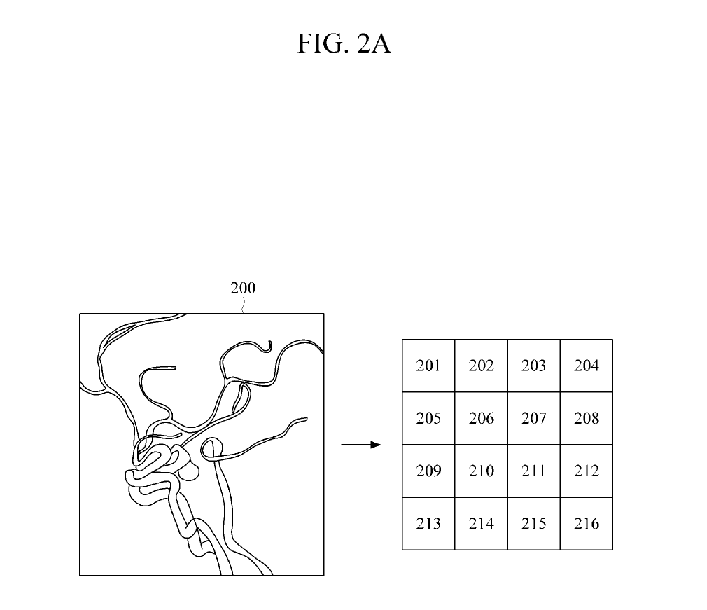

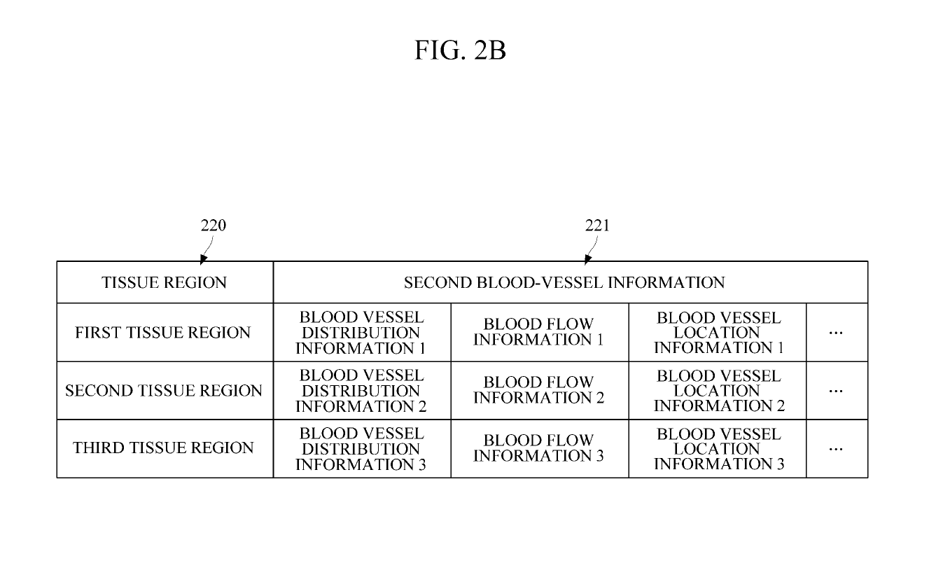

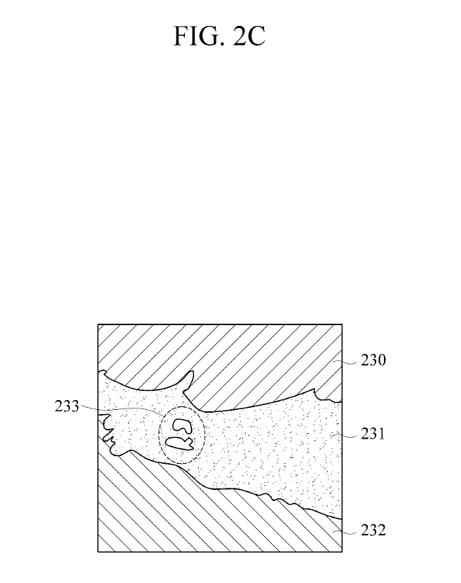

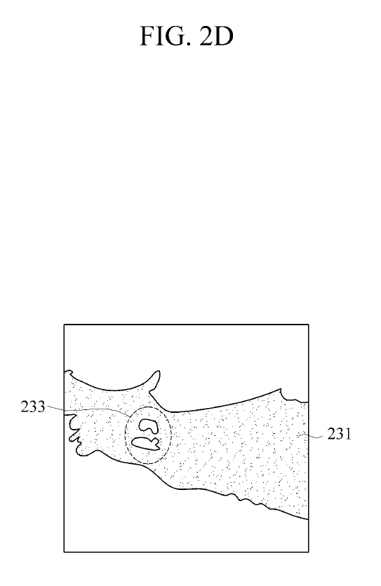

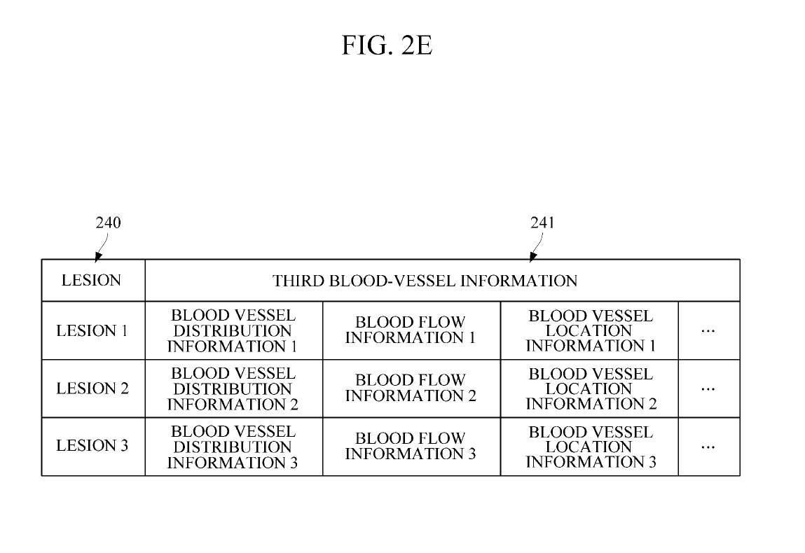

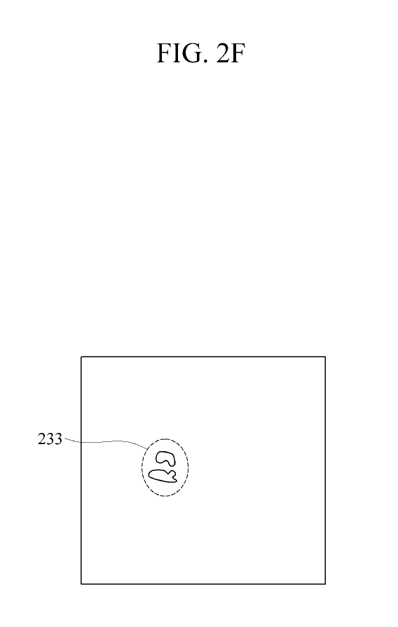

The example assumes that the image is a breast image and a mammary glandular tissue region is extracted among a number of breast tissue areas. The tissue regions may include a subcutaneous fat tissue region, a mammary glandular tissue region, and a pectoralis muscle region. The extraction unit 113 may compare the first blood-vessel information, which is acquired by the acquisition unit 111, with the second blood-vessel information present in the storage unit 112 regarding each breast tissue region, and extract at least one breast tissue region based on the comparison result. Procedures of extracting a tissue region will be described later in detail with reference to FIGS. 2A to 2F.

The setting unit 114 may set at least one of the tissue regions extracted by the extraction unit 113 as a lesion detection target region. A region that has a high probability of having a presence of a lesion may be set as the lesion detection target region by a user, etc. For example, if a user, a manufacturer, etc. sets a cerebellum region or a muscle region as a lesion detection target region, the setting unit 114 may set a cerebellum region or a muscle region among the extracted tissue regions as the lesion detection target region. In the case of a breast image, a user, a manufacturer, or the like may set a mammary glandular tissue region in which a lesion is frequently found as the lesion detection target region. In this example, the setting unit 114 may be able to set only the mammary glandular tissue region among the extracted tissue regions as the lesion detection target region.

The detection unit 115 may detect a lesion from the tissue regions extracted by the extraction unit 113. For example, the detection unit 115 may compare the first blood-vessel information, which is acquired by the acquisition unit 111, with the third blood-vessel information stored in the storage unit 112 regarding each lesion, and extract a lesion from the extracted tissue region based on the comparison result.

The detection unit 115 may detect a lesion from the lesion detection target region set by the setting unit 114. For example, the detection unit 115 may compare the first blood-vessel information, which is included in the lesion detection target region, with the third blood-vessel information stored in the storage unit 112 regarding each lesion, and detect a lesion from the lesion detection target region. Procedures of detecting a lesion will be described in detail later with reference to FIGS. 2A to 2F.

FIGS. 2A to 2F are diagrams illustrating an example explaining how a lesion diagnosis apparatus 100 detects a tissue region and a lesion.

Referring to FIGS. 1 and 2A, the acquisition unit 111 partitions an image 200 into a plurality of regions 201, 202, 203, 204, 205, 206, 207, 208, 209, 210, 211, 212, 213, 214, 215, and 216 of a predetermined size. The acquisition unit 111 may acquire first blood-vessel information about a blood vessel that is included in each of the partitioned regions 201 to 216. The first blood-vessel information may be a variety of information that relates to blood vessels, such as blood-vessel distribution, a location of each blood vessel, blood flow, and the like.

Referring to FIGS. 1 and 2B, the storage unit 112 stores a number of pieces of second blood-vessel information 221 about a blood vessel included in each of tissue regions 220. The second blood-vessel information includes a variety of information that relates to blood vessels, such as blood-vessel distribution, a location of each blood vessel, blood flow, and the like.

Referring to FIGS. 1 and 2C, the extraction unit 113 extracts a first tissue region 230, a second tissue region 231, and a third tissue region 232 from the image based on the first blood-vessel information acquired by the acquisition unit 111. For example, the second tissue region 231 includes a lesion 233.

The extraction unit 113 may compare the first blood-vessel information of each of the partitioned regions 201 to 216 with the second blood-vessel information about the first tissue region 230, the second tissue region 231, and the third tissue region 232, and extract the first to third tissue regions 230 to 232 based on the comparison results. For example, the extraction unit 113 may compare the first blood-vessel information of a first region 201 with the second blood-vessel information 221, and determine that the first blood-vessel information matches the blood-vessel information of the first tissue region among the second blood-vessel information. Then, the extraction unit 113 may extract the first region 201 as the first tissue region. By repeating the above procedure, the extraction unit 113 may be able to extract the first tissue region 230, the second tissue region 231, and the third tissue region 232 from the image based on the first blood-vessel information acquired by the acquisition unit 111.

Referring to FIGS. 1 and 2D, the setting unit 114 sets one of the extracted first, second, and third tissue regions 230, 231, and 232 as a lesion detection target region. In the case of a breast image, the setting unit 114 sets a mammary glandular tissue region 231 as the lesion detection target region. The mammary glandular tissue region 231 may be a tissue region that has the highest probability of the presence of a lesion.

Referring to FIGS. 1 and 2E, the storage unit 112 stores the third blood-vessel information 241 about a blood vessel that is included in each of types of lesions 240. The third blood-vessel information 241 is a variety of information that relates to a blood vessel, such as blood vessel distribution, blood vessel location information, blood flood, and the like.

Referring to FIGS. 1 and 2F, the detection unit 115 detects a lesion 233 from the lesion detection target region 231 set by the setting unit 114. For example, the detection unit 115 may detect the lesion 233 from the lesion detection target region 231 by comparing the first blood-vessel information of the partitioned regions 201 to 216 that correspond to the lesion detection target region 231 with the third blood-vessel information 241 about each of the types of lesions 240 that is stored in the storage unit 112. As a further example, the detection unit 115 may compare the first blood-vessel information of a fifth region 205 with the third blood-vessel information 241, and determine that the first blood-vessel information matches blood-vessel information of the first of the types of lesions 240 among the third blood-vessel information 241. Thereafter, the detection unit 115 may detect the fifth region 205 as a region that includes the lesion 233.

As a result, the detection unit 115 may be able to detect a lesion 233 as a first of the types of lesions 240 from the lesion detection target region 231. In this example, the detected lesion 233 may be identified among the first, the second, and the third of the types of lesions 240.

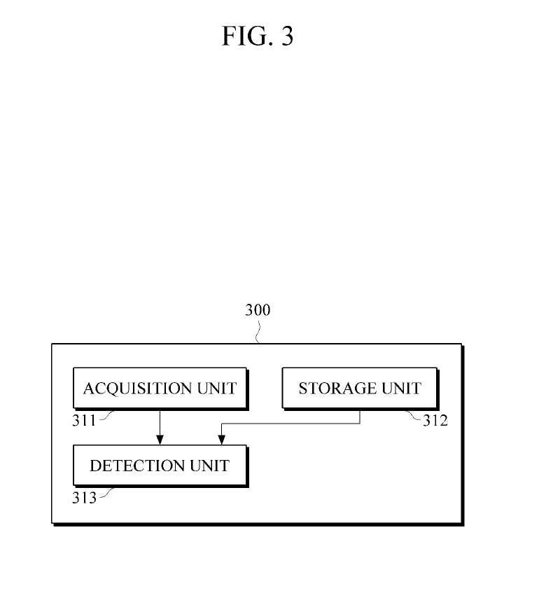

FIG. 3 is a diagram illustrating another example of an apparatus 300 for diagnosing a lesion.

Referring to FIG. 3, apparatus 300 includes an acquisition unit 311, a storage unit 312, and a detection unit 313.

The acquisition unit 311 may acquire first blood-vessel information from an image including a blood vessel.

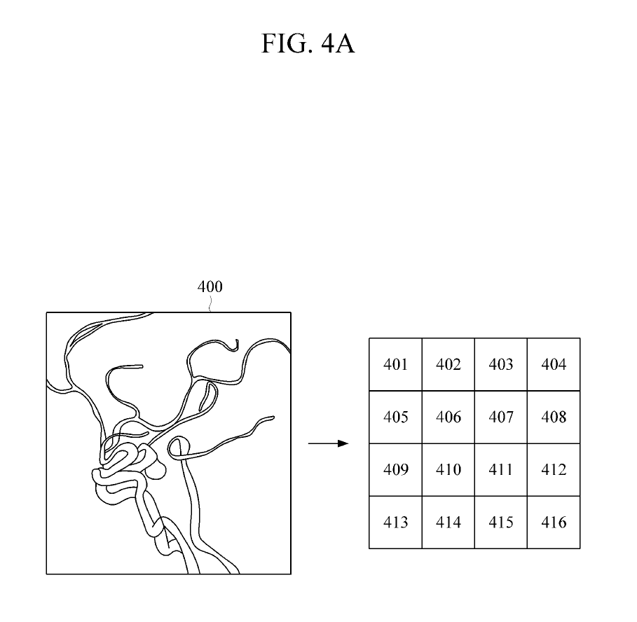

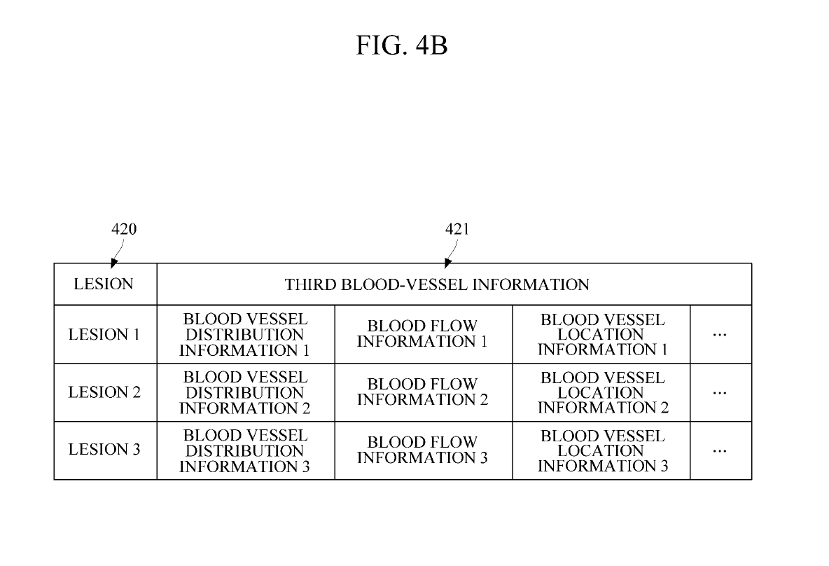

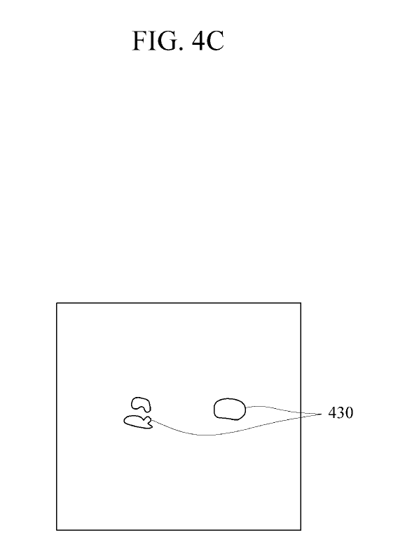

The storage unit 312 may store third blood-vessel information about a blood vessel of each type of lesion (illustrated in FIG. 4B as 420). For example, the storage unit 312 may store blood-vessel information about a blood vessel that is included in a first type of lesion, blood-vessel information about a blood vessel that is included in a second type of lesion, and the like.

The detection unit 313 may detect a lesion from the image by comparing the first blood-vessel information acquired by the acquisition unit 311 with the third blood-vessel information present in the storage unit 112.

FIGS. 4A, 4B, and 4C are diagrams illustrating an example explaining how a lesion diagnosis apparatus 300 detects a tissue region and a lesion.

Referring to FIGS. 3 and 4A, the acquisition unit 311 partitions an image 400 including a blood vessel into a plurality of regions 401, 402, 403, 404, 405, 406, 407, 408, 409, 410, 411, 412, 413, 414, 415, and 416 of a predetermined size. The acquisition unit 311 may acquire a number of pieces of first blood-vessel information about a blood vessel that is included in each of the partitioned regions 401 to 416.

Referring to FIGS. 3 and 4B, the storage unit 312 stores third blood-vessel information 421 about a blood vessel that is included in each of types of lesions 420. For example, the storage unit 312 may store blood-vessel information about a blood vessel that is included in a first type of lesion, blood-vessel information about a blood vessel that is included in a second type of lesion, and the like.

Referring to FIGS. 3 and 4C, the detection unit 313 detects a lesion 430 by comparing the first blood-vessel information regarding each of the partitioned regions 401 to 416 with the third blood-vessel information 421 stored in the storage unit 312 regarding each type of lesion 420. For example, the detection unit 313 may compare the first blood-vessel information of a first region 401 with the third blood-vessel information 421, and determine that the first blood-vessel information matches blood-vessel information of a first type of lesion 420 among the third blood-vessel information 421. Then, the detection unit 313 may detect the first region 401 as a region that includes the lesion 430.

As a result, the detection unit 313 may be able to detect the lesion 430 as a first of the types of lesions 420 from the partitioned regions 401 to 416 of the image 400. At this time, the detected lesion 430 may be identified among the first, the second, and the third of the types of lesions 420.

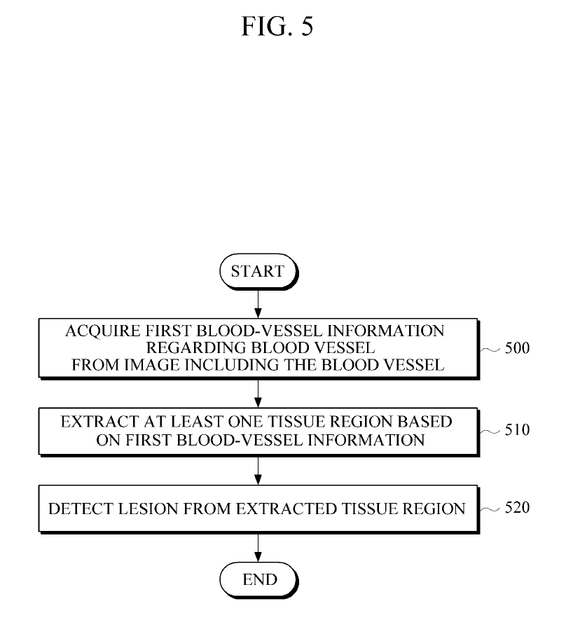

FIG. 5 is a flowchart illustrating an example of a method for diagnosing a lesion. Referring to FIG. 5, an apparatus for diagnosing a lesion acquires first blood-vessel information from an image including a blood vessel at 500. The apparatus extracts at least one tissue region from the image based on the first blood-vessel information at 510.

For example, the apparatus may extract the tissue region by comparing the acquired first blood-vessel information with second blood-vessel information stored in a storage unit regarding each tissue region.

If the image is a breast image, the apparatus may extract a mammary glandular tissue region from the image based on the first blood-vessel information.

The apparatus detects a lesion from the extracted tissue region at 520.

For example, the apparatus may detect the lesion from the tissue region by comparing the first blood-vessel information with third blood-vessel information stored in the storage unit regarding each type of lesion.

In another example, the apparatus may set at least one of the tissue regions as a lesion detection target region. The apparatus may detect a lesion from the lesion detection target region.

According to the teachings above, there is provided an apparatus for diagnosing a lesion that may be able to precisely extract a tissue region and detect a lesion by using blood-vessel information. In addition, the apparatus may increase the probability of precisely detecting a lesion by detecting the lesion in a legion detection target region that has a high probability of the presence of a lesion, and at the same time thereby reduce the time taken to detect the lesion.

The apparatus for diagnosing a lesion may be able to precisely extract a tissue region and detect a lesion using the blood-vessel information, thereby extracting a precise tissue region and detecting a lesion precisely by using blood-vessel information.

In addition, the apparatus may increase the probability of precisely detecting a lesion by detecting the lesion in a legion detection target region that has a high probability of the presence of a lesion, and at the same time thereby reduce the time taken to detect the lesion.

Further, the apparatus may reduce a time taken to detect a lesion by directly detecting a lesion from the image acquired by the acquisition unit.

The methods and/or operations described above may be recorded, stored, or fixed in one or more computer-readable storage media that includes program instructions to be implemented by a computer to cause a processor to execute or perform the program instructions. The media may also include, alone or in combination with the program instructions, data files, data structures, and the like. Examples of computer-readable storage media include magnetic media, such as hard disks, floppy disks, and magnetic tape; optical media such as CD ROM disks and DVDs; magneto-optical media, such as optical disks; and hardware devices that are specially configured to store and perform program instructions, such as read-only memory (ROM), random access memory (RAM), flash memory, and the like. Examples of program instructions include machine code, such as produced by a compiler, and files containing higher level code that may be executed by the computer using an interpreter. The described hardware devices may be configured to act as one or more software modules in order to perform the operations and methods described above, or vice versa. In addition, a computer-readable storage medium may be distributed among computer systems connected through a network and computer-readable codes or program instructions may be stored and executed in a decentralized manner. The program instructions, that is, software, may be distributed over network coupled computer systems so that the software is stored and executed in a distributed fashion. For example, the software and data may be stored by one or more computer-readable storage mediums. Also, functional programs, codes, and code segments for accomplishing the example embodiments disclosed herein can be easily construed by programmers skilled in the art to which the embodiments pertain based on and using the flow diagrams and block diagrams of the figures and their corresponding descriptions as provided herein. Also, the described unit to perform an operation or a method may be hardware, software, or some combination of hardware and software. For example, the unit may be a software package running on a computer or the computer on which that software is running.

A number of examples have been described above. Nevertheless, it should be understood that various modifications might be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. An apparatus for diagnosing a lesion, comprising:
an acquisition unit configured to acquire first blood-vessel information regarding a blood vessel from an image including the blood vessel; and
an extraction unit configured to extract one or more tissue regions from the image based on the first blood-vessel information.

2. The apparatus of claim 1, wherein the extraction unit is further configured to compare the acquired first blood-vessel information with second blood-vessel information from storage to determine the one or more tissue regions to be extracted, the second blood-vessel information being blood-vessel information concerning a plurality of types of tissue regions.

3. The apparatus of claim 1, wherein, if the image is a breast image, the one or more extracted tissue regions comprises a subcutaneous fat tissue region, a mammary glandular tissue region, a pectoralis muscle region, or any combination thereof.

4. The apparatus of claim 1, further comprising:
a detection unit configured to detect a lesion from the one or more extracted tissue regions.

5. The apparatus of claim 4, wherein the detection unit is further configured to compare the acquired first blood-vessel information with third blood-vessel information from storage to detect the lesion, the third blood-vessel information being blood-vessel information concerning a plurality of types of lesions.

6. The apparatus of claim 1, further comprising:
a setting unit configured to set one or more of the one or more tissue regions as a lesion detection target region; and
a detection unit configured to detect a lesion from the lesion detection target region.

7. The apparatus of claim 6, wherein, if the image is a breast image, the lesion detection target region comprises a mammary glandular tissue region.

8. The apparatus of claim 1, wherein:
the acquisition unit is further configured to:
partition the image into a plurality of regions of a predetermined size; and
acquire the first blood-vessel information according to the partitioned regions; and
the extraction unit is further configured to compare the acquired first-blood vessel information with second blood-vessel information from storage to determine the one or more tissue regions to be extracted, the second blood-vessel information being blood-vessel information concerning a plurality of types of tissue regions.

9. The apparatus of claim 1, wherein the acquisition unit is further configured to:
partition the image into a plurality of regions of a predetermined size; and
calculate a ratio of blood vessels to an area of each of the partitioned regions as a distribution of the blood vessels.

10. The apparatus of claim 1, wherein the acquisition unit is further configured to:
partition the image into a plurality of regions of a predetermined size; and
calculate a blood flow per unit time within each of the partitioned regions as blood flow information.

11. The apparatus of claim 1, wherein the first blood-vessel information comprises blood-vessel distribution information, blood-vessel location information, blood flow information, or any combination thereof.

12. An apparatus for diagnosing a lesion, comprising:
an acquisition unit configured to acquire first blood-vessel information regarding a blood vessel from an image including the blood vessel; and
a detection unit configured to detect a lesion from the image based on the first blood-vessel information.

13. The apparatus of claim 12, wherein the detection unit is further configured to compare the acquired first blood-vessel information with third blood-vessel information from storage to detect the lesion, the third blood-vessel information being blood-vessel information concerning a plurality of types of lesions.

14. The apparatus of claim 12, wherein the first blood-vessel information comprises blood-vessel distribution information, blood-vessel location information, blood flow information, or any combination thereof.

15. A method for diagnosing a lesion, comprising:
acquiring first blood-vessel information regarding a blood vessel from an image including the blood vessel; and
extracting one or more tissue regions from the image based on the first blood-vessel information.

16. The method of claim 15, wherein the extracting of the one or more tissue regions comprises:
comparing the acquired first blood-vessel information with second blood-vessel information from storage, the second blood-vessel information being blood-vessel information concerning a plurality of types of tissue regions; and
determining, from the comparing, the one or more tissue regions to be extracted.

17. The method of claim 15, wherein the extracting of the one or more tissue regions comprises, if the image is a breast image, extracting a mammary glandular tissue region.

18. The method of claim 15, further comprising:
detecting a lesion from the one or more tissue regions.

19. The method of claim 18, wherein the detecting of the lesion comprises:
comparing the acquired first blood-vessel information with third blood-vessel information from storage, the third blood-vessel information being blood-vessel information concerning a plurality of types of regions; and
detecting, from the comparing, the lesion.

20. The method of claim 15, further comprising:
setting one or more of the one or more tissue regions as a lesion detection target region; and
detecting a lesion from the lesion detection target region.
